(19) United States
(12) Patent Application Publication
(10) Pub. No.: US 2026/0229322 A1
(43) Pub. Date: Aug. 6, 2026

(54) Systems and Methods for Prognostic Covariate Adjustment in Logistic Regression for Randomized Controlled Trial Design

(71) Applicant: Unlearn.AI, Inc., San Francisco, CA (US)

(72) Inventors: Yunfan Li, Walnut Creek, CA (US); Arman Sabbaghi, Martinez, CA (US)

(73) Assignee: Unlearn.AI, Inc., San Francisco, CA (US)

(21) Appl. No.: 19/152,872

(22) PCT Filed: Jan. 31, 2024
(86) PCT No.: PCT/US2024/013850

§ 371 Date: Jul. 31, 2025

Related U.S. Application Data
(60) Provisional application No. 63/482,395, filed on Jan. 31, 2023.

(51) Int. Cl. G16H 10/20

(52) U.S. Cl. CPC G16H 10/20

(57) ABSTRACT

Systems and methods for prognostic covariate adjustment in logistic regression for randomized controlled trial (RCT) design in accordance with embodiments of the invention arc illustrated. One embodiment includes a method for RCT design using prognostic covariatc adjustment in logistic regression models. The. method includes generating a plurality of digital twin distributions for prospective trial, participants, generating a plurality of digital twins based on the generated digital twin distributions, and calculating prognostic scores for each participant based on their generated digital twins. The method further includes optimizing trial design based on the prognostic scores; fitting a logistic regression model to observed data, including the digital twins of the participants, actual outcomes of each participant, and the prognostic scores; and estimating treatment effects based on the fitted model.

### CROSS-REFERENCE TO RELATED APPLICATIONS

The current application claims priority to U.S. Provisional Patent Application No. 63/482,395 entitled “Efficient Prospective Trial Design with Covariate Adjusted Logistic Regression” filed Jan. 31, 2023. The disclosure of U.S. Provisional Patent Application No. 63/482,395 is hereby incorporated by reference in its entirety for all purposes.

### FIELD OF THE INVENTION

The present invention generally relates to clinical trial design and, more specifically, using prognostic covariate adjustment to improve the statistical power and reduce the sample size of clinical trials.

### BACKGROUND

Clinical trial design is a critical component of medical research that aims to assess the safety and efficacy of biomedical or behavioral interventions on humans. Randomized controlled trials (RCTs) are one of the most common methods used to conduct clinical trials. An RCT typically has two groups, namely the treatment group and the control group, where the control group receives either no treatment or a placebo. RCTs involve randomly assigning participants to either group. A participant in an RCT may be assigned to only one group at a given point in time. This randomization ensures that any differences in outcomes between the two groups can be attributed to the proposed treatment being studied rather than other factors. The use of a control group also allows researchers to compare the effects of the treatment against a baseline, thus allowing researchers to define the treatment effect. RCTs are designed to minimize the occurrence of bias in treatment effect inferences due to confounding variables, inter-current events, and other issues, making them an important tool for generating high-quality evidence that can be used to inform clinical practice and improve patient outcomes. A well-designed RCT may provide a reliable indication of not only the trial outcome but also information on possible adverse effects, lack of efficacy, excess efficacy, and other inter-current events of the experiment.

Covariate adjustment is a statistical technique that is commonly used in clinical research and clinical trials to control for the effects of potentially confounding variables. Covariates are any factors that may be associated with the outcome of a study but are not the interventions under investigation in the RCT. Covariate adjustment allows researchers to account for these variables when performing inferences on the treatment effect, thereby increasing the accuracy and reliability of the study results. By adjusting for covariates, researchers can obtain a more accurate estimate of the true effect of the treatment being studied and improve the validity of their conclusions.

### SUMMARY OF THE INVENTION

Systems and methods for prognostic covariate adjustment in logistic regression for randomized controlled trial (RCT) design in accordance with embodiments of the invention are illustrated. One embodiment includes a method for RCT design using prognostic covariate adjustment in logistic regression models. The method includes generating a plurality of digital twin distributions for prospective trial participants, generating a plurality of digital twins based on the generated digital twin distributions, and calculating prognostic scores for each participant based on their generated digital twins. The method further includes optimizing trial design based on the prognostic scores; fitting a logistic regression model to observed data including the digital twins of the participants, actual outcomes of each participant, and the prognostic scores; and estimating treatment effects based on the fitted model.

In a further embodiment, the plurality of digital twin distributions are generated using models trained on historical patient data, wherein the plurality of digital twin distributions generates forecasts for prospective trial participants.

In still another embodiment, the digital twin distributions are Bernoulli distributions.

In a still further embodiment, the prognostic score is an expectation of the digital twin distribution for a prospective trial participant.

In yet another embodiment, the method further includes computing a set of regression coefficient estimates of the logistic regression model based on the prognostic scores.

In a yet further embodiment, the set of regression coefficients includes a treatment indicator coefficient, and a prognostic score coefficient.

In another additional embodiment, the plurality of treatment effects includes risk difference, risk ratio, and odds ratio.

In a further additional embodiment, optimizing trial design further includes reducing the sample size of the plurality of prospective trial participants and increasing the power of the logistic regression model.

In another embodiment again, the prognostic score is calculated based on a set of baseline covariates.

In a further embodiment again, the prognostic score associates a set of baseline covariates with a probability of an event under control for each prospective trial participant.

In still yet another embodiment, the logistic regression model further includes a treatment indicator wherein prospective trial participants are randomly assigned to either a treatment group or a control group.

In still another additional embodiment, the sample size of the plurality of prospective trial participants includes is reduced based on an efficiency factor.

In a still further additional embodiment, the efficiency factor is determined based on a bias factor and an asymptotic relative efficiency of the logistic regression model. In still another embodiment again, the efficiency factor is estimated based on a ratio of Wald test statistics for an unadjusted logistic regression model and an adjusted logistic regression model.

In a yet further additional embodiment, the ratio of Wald test statistics is predicted based on the variance and an expectation of the probability of an event for a prospective trial participant under control.

In yet another embodiment again, the power of the logistic regression model is determined based on an unadjusted Wald test statistic for null hypothesis on a treatment assignment coefficient and an efficiency factor.

In a yet further embodiment again, the plurality of treatment effects is determined based on a combination of Delta method and G-computation.

In another additional embodiment again, a point estimator for each of the plurality of treatment effects can be computed using G-computation.

In a further additional embodiment again, the prognostic scores are calculated based on an external and historical control dataset.

One embodiment includes a non-transitory machine readable medium containing processor instructions for RCT design using prognostic covariate adjustment in logistic regression models, where execution of the instructions by a processor causes the processor to perform a process that includes generating a plurality of digital twin distributions for prospective trial participants, generating a plurality of digital twins based on the generated digital twin distributions, and calculating prognostic scores for each participant based on their generated digital twins, wherein the prognostic score is an expectation of the digital twin distribution for a prospective trial participant. The process further includes optimizing trial design based on the prognostic scores, fitting a logistic regression model to observed data including the digital twins of the participants, actual outcomes of each participant, and the prognostic scores, computing a set of regression coefficient estimates of the logistic regression model based on the prognostic scores, wherein the set of regression coefficients includes a treatment indicator coefficient, and a prognostic score coefficient, and estimating treatment effects based on the fitted model.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which form a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a process for RCT design using prognostic covariate adjustment in logistic regression models in accordance with an embodiment of the invention.

FIG. 2 illustrates the relationships between the power of adjusted models and unadjusted Wald statistics at different power levels in accordance with an embodiment of the invention.

FIG. 3 illustrates a process for inferring causal estimands in accordance with an embodiment of the invention.

FIG. 4 illustrates an example of using generative models to estimate treatment effects in accordance with an embodiment of the invention.

FIG. 5 illustrates a network where processes for RCT design using prognostic covariate adjustment in logistic regression models can be implemented on in accordance with an embodiment of the invention.

FIG. 6 illustrates a RCT design element where processes for RCT design using prognostic covariate adjustment in logistic regression models are implemented on in accordance with an embodiment of the invention.

FIG. 7 illustrates a designing application that executes instructions to design RCTs using prognostic covariate adjustment in logistic regression models in accordance with an embodiment of the invention.

### DETAILED DESCRIPTION

Randomized controlled trials (RCTs) are a type of clinical trial used to evaluate the safety and effectiveness of medical interventions. RCTs involve randomly assigning participants to either a treatment group or a control group, where the control group receives either no treatment or a placebo. A participant in an RCT may be assigned to only one group at a given point in time. RCTs are important in medical research as they provide high-quality evidence to researchers to support valid and unbiased causal inferences on the effects of the treatment being tested. They can be used to identify potential adverse effects, lack of efficacy, excess effects, and other inter-current events that might not have been detected in earlier studies and are important in obtaining regulatory approval of new drugs and treatments.

Researchers involved in RCTs are typically interested in estimating various estimands associated with an RCT. Estimands, such as but not limited to the average treatment effect for the entire population, the effect on a specific sub-group, and/or the intention-to-treat effect, can govern the specific question the RCT can answer. The estimand to be estimated can impact the selection of an inferential method for the RCT. Therefore, in any well-designed RCT, the choice of an inferential method for inferring treatment effects can play a large role in determining whether the RCTs can be successful. Different inferential methods can be used to answer different research questions and have implications for sampling and analysis. They also have different precisions for inferring the treatment effect, and different inferential methods may require larger sample sizes to achieve the same level of statistical power as others.

A key consideration when designing RCTs is to reduce the variance of treatment effect estimators. However, controlling variance in RCTs that involve logistic regression models may not be a straightforward concept to accomplish. When inferring causal estimands in logistic regression models as a way to measure the effectiveness of the proposed treatment, unadjusted logistic regression models may exhibit lower variance than adjusted logistic regression models, but the unadjusted models may have lesser power compared to the adjusted models. This contradiction is referred to as the non-collapsibility of models, which is an important consideration in RCT design. Power refers to the probability of detecting a true effect if it exists, and underpowered studies can lead to a loss of resources and opportunities.

Among the current methods, covariate adjustment is an often utilized tool for researchers to reduce the variance of treatment effect estimators. In clinical trials, covariate adjustment involves analyzing data through a regression model that includes the treatment indicator and covariates associated with the outcome. Covariate adjustment allows researchers to control for the covariates, which may be potentially confounding variables associated with outcomes in an RCT. By adjusting for these variables, researchers can isolate the effect of the treatment under investigation, which results in a more accurate estimator of the true effect of the treatment. Furthermore, by helping to explain the variation in the outcomes under treatment and control, covariate adjustment can improve the statistical power or reduce the required sample size to achieve a desired power for the study. Covariate adjustment is also considered to be a more efficient method to decrease the variance of the treatment effect estimator or improve the power of the test for the treatment effect compared to increasing the sample size, which can be costly and time-consuming.

Covariate adjustment, however, has been limited to RCTs adopting a linear regression model, as it can be difficult to apply covariate adjustment to RCTs that utilize other types of regression models. In particular, covariate adjustment in logistic regression for binary outcomes presents several unsolved difficulties. When applying covariate adjustment to logistic regression models, complications arise due to the noncollapsibility of certain estimands in logistic regression models. Noncollapsibility occurs when the treatment effect estimand changes based on which covariates are included in a logistic regression model. Noncollapsibility makes it difficult to interpret the effects of treatment when adjusting for covariates using logistic regression models, as these adjustments can change the meaning of both the treatment effect estimand and the variance of its estimator compared to the unadjusted logistic regression model.

Systems and methods in accordance with many embodiments of the invention can extend the application of covariate adjustment into logistic regression models. In many embodiments, systems and methods utilize a non-confounding predictive covariate, referred to as a prognostic score, to adjust logistic regression models. In numerous embodiments, systems and methods can increase the power of tests and/or decrease the sample size necessary to maintain the power in logistic regression. In several embodiments, systems and methods can generate digital twins that represent prospective trial participants using historical data from other trials that have already been conducted. In many embodiments, digital twins are generated using models trained on historical patient data and then applied to compute forecasts for prospective new patients. Digital twins can effectively serve as a forecast of a person's health in the future. By using digital twins that represent prospective trial participants, systems and methods are able to determine the amount of power increase and/or sample size reduction as early as in the design stage of RCTs. This can save valuable costs and time expenditures that may otherwise be necessary when the trial is being conducted. In some embodiments, the inclusion of non-confounding predictive covariates is capable of refining RCT design even when a model is incorrectly specified.

In numerous embodiments, systems and methods are capable of evaluating important causal estimands that quantify the effectiveness of RCTs. Causal estimands such as but not limited to risk difference (RD), relative risk (RR), and/or odds ratio (OR) may be defined based on the Neyman-Rubin causal model. In the context of RCT design, participants are chosen from a hypothetical infinite population known as a super-population. The super-population OR is generally inherently noncollapsible, whereas the super-population RD and RR may be collapsible. As certain super-population treatment effect estimands change based on which covariates are included in the logistic regression model due to its noncollapsibility, it introduces complexity in conducting covariate adjustment. Noncollapsibility will be discussed in detail further below. In numerous embodiments, systems and methods can adjust logistic regression models such that the treatment effect estimators can have increased precision compared to unadjusted logistic regression models.

For each participant i=1, . . . , N in the RCT, their treatment assignment may be denoted as wᵢ∈{0, 1}, where 0 indicates an assignment to the control group and 1 indicates an assignment to the treatment group. Each participant can only receive one treatment level. xᵢ∈ [symbol] ^{L} may denote the covariate vector for each participant. In numerous embodiments, a participant's covariate vector contains a plurality of characteristics that are observed either prior to treatment assignment or after treatment assignment and are known to be unaffected by treatment. The possible values of an outcome that is a binary endpoint can be denoted by 0 and 1, with 1 indicating an event. RCT designs typically rely on the Stable Unit Treatment Value Assumption (SUTVA), which provides that each combination of participant and treatment assignment corresponds to a well-defined binary outcome, and the outcome for a participant does not depend on treatments assigned to others. The potential outcome for participant i under treatment w can be defined by Yᵢ(w).

Evaluating the effectiveness and efficacy of new treatments that are tested in an RCT generally involves inferring the finite-population causal estimands on the participants in the RCT, each of which is defined via a comparison between {Yᵢ(1): i=1, . . . , N} versus the {Yᵢ(0): i=1, . . . , N}. One estimand is the average treatment effect

$$\overline{Y} ( 1 ) - \overline{Y} ( 0 ) = \frac{1}{N} {\sum }_{i = 1}^{N} {Y}_{i} ( 1 ) - \frac{1}{N} {\sum }_{i = 1}^{N} {Y}_{i} ( 0 ) ,$$

which can also be referred to as the risk difference for the binary endpoint and is denoted by Δ_{RD}. Two other estimands for binary endpoints are the relative risk Δ_{RR}=Y̅(1)/Y̅(0) and the odds ratio Δ_{OR}=[Y̅(1)/{1−Y̅(1)}]/[Y̅(0)/{1−Y̅(0)}]. For the latter two estimands, it can be assumed that Y̅(w)≠0, 1 for w∈{0, 1}.

In an RCT based on the Neyman-Rubin causal model, causal inference may be regarded as a missing data problem, with at most one observed potential outcome for any participant. The treatment assignment mechanism, which may also be known as the probability mass function p(w₁, . . . , w_{N}|Y₁(0), . . . , Y_{N}(0), Y₁(1), . . . , Y_{N}(1), x₁, . . . , x_{N}), is effectively a missing data mechanism. Three important conditions for a treatment assignment mechanism such that the RCT remains regular are that it is unconfounded (i.e., there are no lurking confounders associated with both treatment assignment and the potential outcomes conditional on the covariates), probabilistic, and individualistic (i.e., a participant's treatment assignment does not depend on the covariates or potential outcomes of others). Violations of these conditions would introduce complications in the design and analysis of an RCT. The completely randomized design is an assignment mechanism for RCTs that satisfies these conditions.

### Prognostic Covariate Adjustment in Logistic Regression

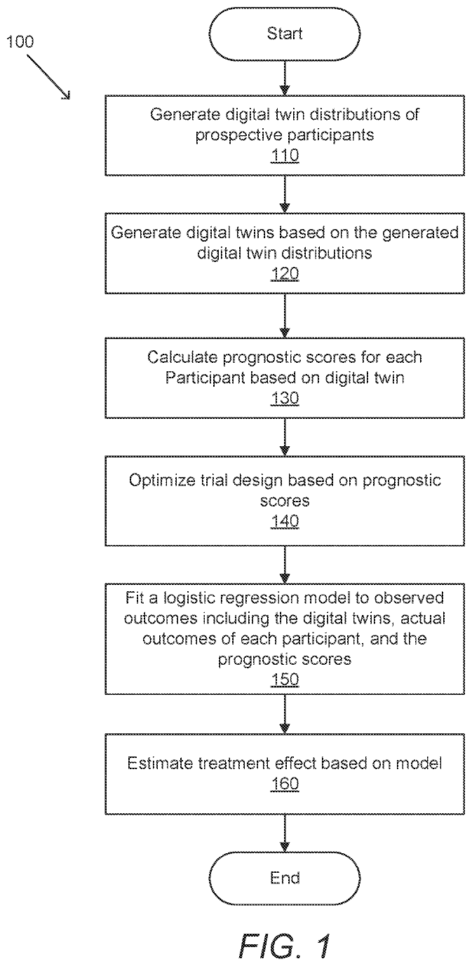

Systems and methods in accordance with many embodiments can infer causal estimands such as but not limited to risk difference (RD), relative risk (RR), and odds ratio (OR) by adjusting for a single covariate in logistic regression. In many embodiments, a single predictor variable, which is a prognostic score, is included to adjust logistic regression models. A process for RCT design using prognostic covariate adjustment in logistic regression models in accordance with an embodiment of the invention is illustrated in FIG. 1. Process 100 generates (110) a plurality of digital twin distributions of prospective trial participants. In many embodiments, the digital twin distributions for participants i=1, . . . , N at a specified time-point is a Bernoulli distribution for their potential outcome under control, with the probability mᵢ of an event being a function of their covariate vector xᵢ∈ [symbol] ^{L}.

Process 100 computes (120) a plurality of digital twins based on the generated digital twin distributions. In many embodiments, digital twins are generated using models trained on historical patient data and then applied to compute forecasts for new patients. In several embodiments, a digital twin is generated for each prospective trial participant.

Process 100 calculates (130) prognostic scores for each participant based on their generated digital twins. In numerous embodiments, prognostic scores are defined for each participant as the expectation of their respective digital twin distribution. Prognostic scores can effectively capture the associations between the baseline covariates of each participant and the probability of an event under control. Prognostic scores can satisfy requirements set forth in regulatory guidance documents on covariate adjustment that recommend a small number of covariates for adjustment. Prognostic scores in accordance with some embodiments can be calculated based on external, historical data sets.

Process 100 optimizes (140) trial design based on the prognostic scores. In several embodiments, trial design may be optimized by performing sample size reduction and/or power gain calculations based on the adjusted logistic regression model.

Process 100 fits (150) a logistic regression model to observed data including the digital twins, actual outcomes of each participant, and the prognostic scores. In several embodiments, fitting a logistic regression model allows for the computing of regression coefficients of the model. Fitting a logistic regression model that adjusts solely for the prognostic score instead of the high-dimensional covariate vector can liberate degrees of freedom in the model. In several embodiments, a logistic regression model that adjusts for the log-odds transformation of the prognostic scores may be fitted to the observed outcomes. Logistic regression will be discussed in detail further below. In many embodiments, actual outcomes are outcomes predicted based on the generated digital twins in an RCT.

Process 100 estimates (160) treatment effects based on the fitted model. In many embodiments, treatment effects, such as but not limited to risk difference, relative risk, and/or odds ratio can be estimated based on the adjusted model. These treatment effect estimands can indicate the difference due to treatments for prospective trial participants.

The prognostic score for participant i is defined as mᵢ. It can be calculated prospectively in an RCT prior to any treatment assignments. Specifically, artificial intelligence (AI) algorithms can be used to specify a functional form for mᵢ in terms of baseline covariates based on historical control data. The functional form can be implemented by any mathematical or computational means. AI algorithms are particularly powerful in this context because they can effectively capture associations between baseline predictors and the probability of an event under control. The use of historical control data for modeling and validating the prognostic score can help eliminate additional model selection steps in logistic regression modeling that would complicate the analysis of an RCT.

In some embodiments, the baseline covariates are the sole inputs for a model that specifies a digital twin distribution. Therefore, the prognostic score itself can be a covariate that can be incorporated as a predictor in logistic regression. The adjusted logistic regression model can be specified as

$$P r ( {y}_{i} = 1 \vert {w}_{i} , {m}_{i} ) = \frac{exp ( {β}_{0} + {β}_{1} {w}_{i} + {β}_{2} {m}_{i} )}{1 + exp ( {β}_{0} + {β}_{1} {w}_{i} + {β}_{2} {m}_{i} )} . \qquad ( 1 )$$

The inclusion of the prognostic score in model (1) can enable two potential advantages over an unadjusted model. First, the necessary sample size such that the power of the test for H₀: β₁=0 is the same as the power of the test for

$${H}_{0}^{*} : {β}_{1}^{*} = 0$$

in accordance with several embodiments can be reduced, where

$${β}_{1}^{*}$$

and β₁ parameters are referred to as “treatment effects”, with

$${β}_{1}^{*}$$

being an unconditional treatment effect estimand and β₁ being an conditional treatment effect estimand. Second, adjusted models can boost the power of the test for H₀: β₁=0 compared to that of the test for

$${H}_{0}^{*} : {β}_{1}^{*} = 0$$

for a fixed sample size. In addition to considerations of sample size reductions and power boosts for tests of the treatment indicator coefficient in logistic regression, systems and methods in accordance with several embodiments utilize a combination of g-computation with the adjusted model (1) to improve the precision of inferences for Δ_{RD}, Δ_{RR}, and Δ_{OR}. In selected embodiments where the logistic regression model adjusts for the log-odds transformation of the prognostic scores, the adjusted logistic regression model can be specified as

$$P r ( {y}_{i} = 1 \vert {w}_{i} , {m}_{i} ) = \frac{exp ( {β}_{0} + {β}_{1} {w}_{i} + {β}_{2} log \{ {m}_{i} / ( 1 - {m}_{i} ) \} )}{1 + exp ( {β}_{0} + {B}_{1} {w}_{i} + {β}_{2} log \{ {m}_{i} / ( 1 - {m}_{i} ) \} )} . \qquad ( 2 )$$

While specific processes for RCT design using prognostic covariate adjustments in logistic regression models are described above, any of a variety of processes can be utilized to perform prognostic covariate adjustments as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Logistic Regression

Logistic regression is an established methodology for modeling the probability of an event with a binary endpoint as a function of predictor variables. The unknown probabilities of potential outcomes for participants in each group of an RCT Pr{Yᵢ(1)=1|xᵢ} and Pr{Yᵢ(0)=1|xᵢ} can be modeled based on the observed outcomes and the application of the standard logistic function to the dot product of a vector of predictors vᵢ∈ [symbol] ^{K} (defined based on the wᵢ and xᵢ) and unknown regression coefficients β=(β₀, . . . , β_{K-1})^{T}∈ [symbol] ^{K}. Traditional inferences on the super-population odds ratio estimand via logistic regression involved inferences for the entry in β corresponding to wᵢ. Under the Neyman-Rubin Causal Model, inferences on risk difference Δ_{RD}, relative risk Δ_{RR}, and/or odds ratio Δ_{OR} can be performed by combining logistic regression with either multiple imputations of missing potential outcomes or g-computation.

The logistic regression model may be fitted via maximum likelihood to the observed outcomes yᵢ, which are functions of the treatment assignments and potential outcomes defined by yᵢ=wᵢYᵢ(1)+(1−wᵢ)Yᵢ(0). The general form of the model is

$$P r ( {y}_{i} = 1 \vert {v}_{i} ) = exp ( {v}_{i}^{T} β ) / \{ 1 + exp ( {v}_{i}^{T} β ) \} .$$

All potential outcomes can be assumed to be mutually independent conditional on the predictors. The likelihood function can be represented by

$$L ( β ) = {\prod }_{i = 1}^{N} exp ( {w}_{i} {v}_{i}^{T} β ) {\{ 1 + exp ( {v}_{i}^{T} β ) \}}^{- 1} .$$

Assuming that there is no complete separation or quasi-complete separation, that the endpoint values are not sparse, and that there is no perfect collinearity in the matrix of predictor vectors

$$V = ( \begin{matrix}{v}_{1}^{T} \\ ⋮ \\ {v}_{N}^{T}\end{matrix} ) ,$$

maximum likelihood-based inferences can be performed for logistic regression.

The interpretation of the entries in β may depend on the predictors in vᵢ. For an example model vᵢ=(1, wᵢ)^{T}, which corresponds to the unadjusted logistic regression model, the entries in β may be denoted by

$${β}_{0}^{*} and {β}_{1}^{*} and exp ( {β}_{0}^{*} )$$

be interpreted as the odds of an event under control and exp

$$( {β}_{1}^{*} )$$

can be interpreted as we multiplicative change in the odds of an event under treatment compared to control, where exp

$$( {β}_{1}^{*} )$$

is a super-population odds ratio estimand. The unadjusted logistic regression model in this case may be defined as:

$$Pr ( {y}_{i} = 1 \vert {v}_{i} ) = \frac{exp ( {β}_{0}^{*} + {β}_{1}^{*} {w}_{i} )}{1 + exp ( {β}_{0}^{*} + {β}_{1}^{*} {w}_{i} )} . \qquad ( 3 )$$

The interpretations of β may differ from the case in which additional covariates are included in vᵢ. To illustrate, consider the case in which the predictor vector vᵢ=(1, wᵢ, xᵢ)^{T} includes a covariate xᵢ∈ [symbol] in addition to the treatment assignment. This can be referred to as an adjusted logistic regression model, where entries in β can be denoted by β⁰, β₁, and β₂. In this case, the model can be specified by:

$$Pr ( {y}_{i} = 1 \vert {v}_{i} ) = \frac{exp ( {β}_{0} + {β}_{1} {w}_{i} + {β}_{2} {x}_{i} )}{1 + exp ( {β}_{0} + {β}_{1} {w}_{i} + {β}_{2} {x}_{i} )} \qquad ( 4 )$$

where exp(β₀) is the odds of an event under control when xᵢ=0, and exp(β₁) is the multiplicative change in the odds of an event under treatment compared to control that is defined conditional on xᵢ. This may differ from the ratio of the marginal odds for the treatment and control groups that is calculated by averaging over the distribution of the covariate xᵢ.

The

$${β}_{1}^{*}$$

and β₁ parameters in equations (3) and (4) have traditionally been referred to as “treatment effects” for the unadjusted and adjusted models, respectively. Under the Neyman-Rubin Causal Model, these are not valid finite-population treatment effects as they do not involve the potential outcomes for the participants. Furthermore, these parameters may have different interpretations and magnitudes, because

$${β}_{1}^{*}$$

is defined without consideration of the covariates whereas β₁ is defined conditional on xᵢ. However, this issue may be addressed by defining treatment effects for binary endpoints in a manner that is agnostic to the logistic regression model specification by considering the finite-population estimands Δ_{RD}, Δ_{RR}, and Δ_{OR}

### Noncollapsibility in Logistic Regression

The phenomenon in which the super-population treatment effect estimand changes based on which covariates are included in the logistic regression model is referred to as noncollapsibility. The super-population odds ratio estimand is noncollapsible, whereas the risk difference and relative risk estimands are collapsible.

Noncollapsibility in logistic regression can be illustrated by comparing an unadjusted model with an adjusted model. The adjusted model is generally of more importance in practice, but noncollapsibility can lead to the precision of the estimated odds ratio from this model being less than that of the former model. The standard deviation for 31 from model (4) could be greater than that for

$$\text{?}$$

from model (3). This inequality in the precisions of the estimators is difficult to reconcile because the Wald test for H₀: β₁=0 under logistic regression with covariate adjustment could have more power than that for H₀:

$${β}_{1}^{*} = 0$$

without covariate adjustment. This appears to contradict the intuition from linear regression (which is collapsible), in which covariate adjustment both reduces the standard error for the coefficient estimator and increases the power for testing the regression coefficient associated with the treatment assignment.

This paradox can be explained by the fact that the two “treatment effects” for models (3) and (4) differ in both nature and magnitude, with the magnitude of the conditional odds ratio generally being greater than that for the unconditional odds ratio. Power discrepancy can also be resolved by realizing that when there is no treatment effect, the population (marginal) and the subgroup (conditional) odds ratios both equal 1, and the odds ratio is strictly collapsible.

Coefficient estimators across the logistic regression models specified in equations (3) and (4) can be compared by calculating the asymptotic bias factor and asymptotic relative efficiency (ARE) of

$$\text{?}$$

versus \hat{β}₁. These quantities can effectively evaluate the consequences of omitting a covariate that is associated with the outcome, or alternatively, of inferring the super-population odds ratio estimand based on the unadjusted model when the adjusted model is more appropriate. This comparison is formulated in terms of the limiting case β₁→0, which is relevant for the hypothesis test H₀: β₁=0. The comparison establishes that both the bias factor and ARE of

$$\text{?}$$

are functions of β₀, β₂, and xᵢ, but not of β₁.

To formally define the bias factor and ARE for

$$\text{?}$$

versus \hat{β}₁, function

$${β}_{1}^{*} : {ℝ}^{3} \to ℝ$$

may be defined, under abuse of notation, in terms of the adjusted model (4) and its parameters β₀, β₁, and β₂ by integrating over the distribution of the covariate xᵢ. In many embodiments, the bias factor is defined as

$${lim}_{{β}_{1} \to 0} \frac{\partial }{\partial {β}_{1}} \{ {β}_{1}^{*} ( {β}_{0} , {β}_{1} , {β}_{2} ) \} , \qquad ( 5 )$$

and captures the size of the treatment assignment coefficient under the misspecified, unadjusted model compared to the coefficient under the correctly specified, adjusted model near the value of 0. As the bias factor considers only the case of β1→0, the linear term of the Taylor expansion of

$${β}_{1}^{*} ( {β}_{0} , {β}_{1} , {β}_{2} )$$

about β₁=0 would be sufficient to facilitate the calculation of equation (5). The bias factor, can be determined by:

$${lim}_{{β}_{1} \to 0} \frac{\partial }{\partial {β}_{1}} \{ {β}_{1}^{*} ( {β}_{0} , {β}_{1} , {β}_{2} ) \} = 1 - \frac{Var ( {μ}_{0 , i} )}{E ( {μ}_{0 , i} ) \{ 1 - E ( {μ}_{0 , i} ) \}} , \qquad ( 6 )$$

where μ_{0,i}=exp(β₀+β₂xᵢ)/{1+exp(β₀+2xᵢ)} denotes the predictive probability of an event for participant i under control. If the variance of xᵢ is 0, or if β₂=0, then

$${β}_{1} = {β}_{1}^{*}$$

as expected. Furthermore, larger variances in xᵢ and/or larger values of β₂ may increase the bias factor.

The ARE of

$$\text{?}$$

versus \hat{β}ᵢ, can be defined by:

$$A R E ( \text{?} , {\hat{β}}_{1} ) = {[ {lim}_{{β}_{1} \to 0} \{ \frac{\partial {β}_{1}^{*}}{\partial {β}_{1}} \} {\{ \frac{\partial {β}_{1}}{\partial {β}_{1}} \}}^{- 1} ]}^{2} [ {lim}_{{β}_{1} \to 0} \frac{Var ( {\hat{β}}_{1} )}{Var ( \text{?} )} ] . \qquad ( 7 )$$

Under the null hypothesis and by virtue of the independence of treatment assignment and the covariate, expressions for Var(\hat{β}₁) and

$$Var ( {\hat{β}}_{1}^{*} )$$

can be obtained that involve only the expectation and variance of μ_{0,i}. Specifically, the ARE for logistic regression may be defined as

$$A R E ( \text{?} to {\hat{β}}_{1} at {β}_{1} = 0 ) = 1 - \frac{Var ( {μ}_{0 , i} )}{E ( {μ}_{0 , i} ) \{ 1 - E ( {μ}_{0 , i} ) \}} , \qquad ( 8 )$$

which is equivalent to equation (6). The ARE is generally less than 1 when Var(μ_{0,i})>0, so the odds ratio estimated from the unadjusted model has a smaller variance than the odds ratio estimated from the adjusted model.

In many embodiments, g-computation is utilized to infer Δ_{RD}, Δ_{RR}, and Δ_{OR} under the Neyman-Rubin Causal Model. G-computation can be utilized to infer marginal estimands in the case of binary endpoints. It is effectively a “plug-in” estimator that can utilize the maximum likelihood estimators of the logistic regression coefficients to replace all observed and missing potential outcomes in the RCT with their predicted probabilities. G-computation can yield consistent estimators for the interpretable causal estimands in the case of noncollapsibility under the logistic regression model, even in the case of model misspecification. G-computation is typically model agnostic in that it can target estimands that are well-defined in terms of potential outcomes without reference to any specified model. This enables researchers to infer estimands of scientific interest and frees them from selecting estimands based on mathematical convenience or modeling conventions. For example, if the risk difference is pertinent, then Δ_{RD} rather than Δ_{OR} can be the target estimand, and any model can be utilized to infer it via g-computation.

As an illustration of g-computation, consider the application of model (4) to infer the risk difference, relative risk, and odds ratio estimands. Let \hat{β}=(\hat{β}₀, \hat{β}₁, \hat{β}₂)^{T} denote the maximum likelihood estimators for the logistic regression coefficients. For each participant, their probabilities of an event under treatment and control may be estimated by pᵢ (1)=exp(\hat{β}₀+\hat{β}₁+\hat{β}₂xᵢ)/{1+exp(\hat{β}₀+\hat{β}+β₂xᵢ)}, and

pᵢ (0)=exp(\hat{β}₀+\hat{β}₂xᵢ)/{1+exp(\hat{β}₀+\hat{β}₂xᵢ)}, respectively. These estimators can then be used to calculate the averages

$$: ⋅ \overline{p} ( 1 ) = {\sum }_{i = 1}^{N} {p}_{i} ( 1 ) / N \overline{p} ( 0 ) = {\sum }_{i = 1}^{N} {p}_{i} ( 0 ) / 1 ∀ . \qquad ( 1 )$$

In several embodiments, point estimators of Δ_{RD}, Δ_{RR}, and Δ_{OR} can be specified by replacing Y̅(1) by p̅(1) and Y̅(0) by p̅(0) in the definitions of the original estimands, as in plug-in estimation. More formally, [symbol] =p̅(1)−p̅(0), [symbol] =p̅(1)/p̅(0), and [symbol] =[p̅(1)/{1−p̅(1)}]/[p̅(0)/{1−p̅(0)}].

### Sample Size Reduction and Power Gain With Respect to the Treatment Assignment Coefficient

Sample size reductions and power gains from logistic regression models adjusted with prognostic scores can be prospectively estimated by combining two sets of expressions. In several embodiments, the first set includes the Wald test statistics W_{unadj} and W_{adj} for the hypotheses

$${H}_{0}^{*} : {β}_{1}^{*} = 0$$

and H₀: β₁=0 of the treatment assignment coefficients from models (3) and (1), respectively. In some embodiments, the second set consists of the formulae for the bias factor and ARE of

$${\hat{β}}_{1}^{*}$$

versus \hat{β}₁ from equations (6) and (8), respectively. In many embodiments, systems and methods can better inform the design of an RCT for performing hypothesis tests for the treatment effect with respect to a binary endpoint via logistic regression with covariate adjustment.

The ratio of W_{unadj} and W_{adj} can be expressed in terms of the bias factor and ARE for

$${\hat{β}}_{1}^{*}$$

versus \hat{β}₁ to derive the formulae for sample size reductions and power gains. The Wald statistic for testing H₀: θ=0 for an unknown parameter θ is

$$W = {N}^{1 / 2} {\hat{θ}}_{N} {\hat{V}}_{N}^{- 1 / 2} ,$$

where N indicates the sample size. \hat{θ}_{N} is a point estimator of θ such that \hat{θ}_{N} [symbol] θ as N→∞, and \hat{V}_{N}/N is a consistent estimator of the asymptotic variance of \hat{θ}_{N}. The term

$${\hat{V}}_{N}^{- 1}$$

may be interpreted as the average amount of information provided by each observation. In many embodiments, W_{unadj}/W_{adj} is obtained based on the recognition that the bias factor in equation (6) approximates

$${\hat{β}}_{1}^{*} / {\hat{β}}_{1} ,$$

and the ARE in equation (8) approximates the ratio of the variances of

$${\hat{β}}_{1}^{*}$$

and \hat{β}₁. Hence, for fixed N,

$$\frac{{W}_{unadj}}{{W}_{adj}} \approx \frac{{\hat{β}}_{1}^{*} / {\hat{β}}_{1}}{\sqrt{Var ( {\hat{β}}_{1}^{*} ) / Var ( {\hat{β}}_{1} )}} \approx \sqrt{1 - \frac{Var ( {μ}_{0 , i} )}{𝔼 ( {μ}_{0 , i} ) \{ 1 - 𝔼 ( {μ}_{0 , i} ) \}}} , \qquad ( 9 )$$

where μ_{0,i} is defined as in equation (6) but with xᵢ replaced by mᵢ. In certain embodiments where the logistic regression model adjusts for the log-odds transformation of the prognostic scores, xᵢ may be replaced by log {mᵢ/(1−mᵢ)} The expectation and variance in this equation are calculated for the entire population of μ_{0,i} values. The right-hand side of equation (9) may be referred to as the efficiency factor and denoted by f_{EFF}. In general, a smaller value of f_{EFF} is better, as it indicates greater sample size reduction or power gain for models adjusted using prognostic scores compared to the unadjusted models. This factor may decrease as Var(μ_{0,i}) increases for fixed [symbol] (μ_{0,i}), or as [symbol] (μ_{0,i})→0 or [symbol] (μ_{0,i})→1. Under any of these situations, adjustment for the prognostic score can have a larger effect on the Wald test statistic, and hence, the sample size reduction and power gain may be affected more compared to the unadjusted model.

The prospective (total) sample size reduction formula for powering a study with respect to models adjusted with the prognostic score can be derived by solving for N_{adj} when N_{unadj} is fixed in equation (9), which can be derived by utilizing Wald test statistics to yield approximations for power calculations. In particular, the distributions of W_{unadj} and W_{adj} can be approximated by standard Normal distributions under their corresponding null hypotheses, and the powers of the Wald tests for the treatment assignment coefficients in models (3) and (1) can be approximated by

$${ζ}_{unadj} = Φ ( {Φ}^{- 1} ( \frac{α}{2} ) + {W}_{unadj} ) + Φ ( {Φ}^{- 1} ( \frac{α}{2} ) - {W}_{unadj} ) \qquad ( 10 )$$

$$and$$

$${ζ}_{adj} = Φ ( {Φ}^{- 1} ( \frac{α}{2} ) + {W}_{adj} ) + Φ ( {Φ}^{- 1} ( \frac{α}{2} ) - {W}_{adj} ) , \qquad ( 11 )$$

respectively, where α is the Type I error rate (and usually taken as 0.05). Thus, suppose N_{unadj} is identified such that model specified in (3) has power 0.8 for testing H₀:

$${β}_{1}^{*} = 0 .$$

Using the previous points, N_{adj} can be identified such that the model described by equation (1) has power 0.8 for testing H₀: β₁=0 by considering the case of W_{unadj}/W_{adj}≈1 (so that ζ_{unadj}≈ζ_{adj}), and the equation

$$\begin{matrix}\frac{{W}_{unadj}}{{W}_{adj}} & \approx \frac{{\hat{β}}_{1}^{*} \sqrt{{N}_{unadj}} / {\hat{β}}_{1}}{\sqrt{Var ( {\hat{β}}_{1}^{*} ) {N}_{adj} / Var ( {\hat{β}}_{1} )}} \\ & \approx \sqrt{\frac{{N}_{unadj}}{{N}_{adj}} [ 1 - \frac{Var ( {μ}_{0 , i} )}{E ( {μ}_{0 , i} ) \{ 1 - 𝔼 ( {μ}_{0 , i} ) \}} ]} \\ & = {f}_{EFF} \sqrt{\frac{{N}_{unadj}}{{N}_{adj}}}\end{matrix} . \qquad ( 12 )$$

Therefore, the sample size reduction formula for adjusted models compared to the unadjusted analysis is

$${N}_{adj} = {f}_{EFF}^{2} {N}_{unadj} .$$

By utilizing prior point estimates of knowledge of the coefficients in the adjusted model, Var(μ_{0,i}) and [symbol] (μ_{0,i}) in equation (12) may be estimated. It is important to recognize that the magnitudes of Δ_{RD}, Δ_{RR}, and Δ_{OR} are not involved in this calculation, and that this approach can be implemented based solely on historical information.

In numerous embodiments, the formula for the power gain of adjusted models compared to the unadjusted models can be derived by first approximating W_{adj} as a function of W_{unadj} and f_{EFF} based on equation (9), and then incorporating that approximation into the power calculation in equation (11). More formally, for a fixed sample size N, W_{adj}≈W_{unadj}/f_{EFF} can be approximated:

$${ζ}_{adj} \approx Φ ( {Φ}^{- 1} ( \frac{α}{2} ) + \frac{{W}_{unadj}}{{f}_{EFF}} ) + Φ ( {Φ}^{- 1} ( \frac{α}{2} ) - \frac{{W}_{unadj}}{{f}_{EFF}} ) . \qquad ( 13 )$$

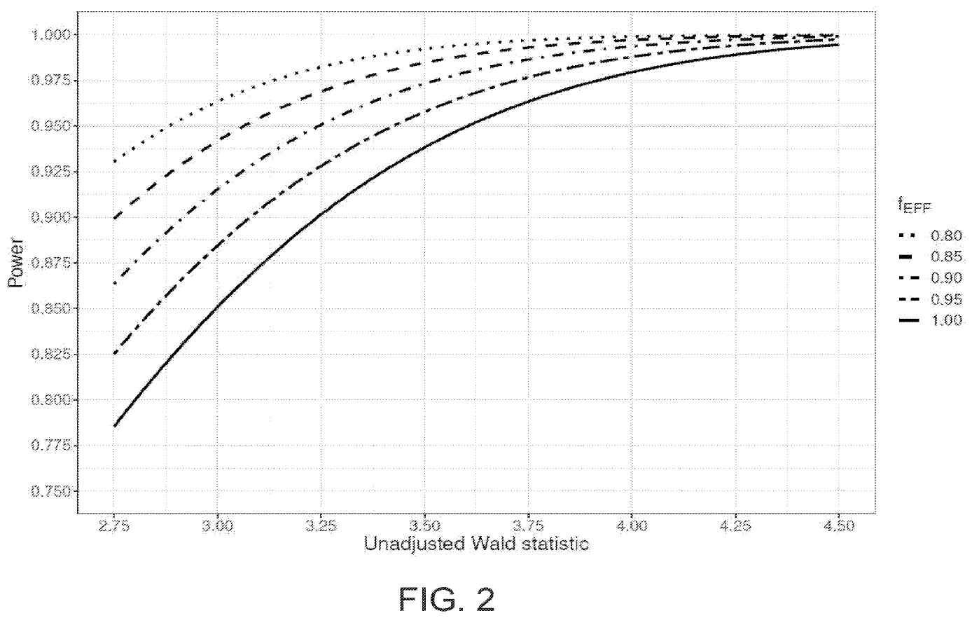

This approximation indicates that the power gain ζ_{adj}-ζ_{unadj} may depend both on the unadjusted Wald test statistic (alternatively, the unadjusted power ζ_{unadj}) and f_{EFF}. FIG. 2 illustrates the relationships between the power of adjusted models and unadjusted Wald statistics at different power levels in accordance with an embodiment of the invention. FIG. 2 visualizes the relationships between the power of an adjusted model, W_{unadj}, and f_{EFF} via five power curves that correspond to f_{EFF}=0.8, 0.86, 0.9, 0.95, 1. In this figure, the range of the y-axis corresponds to the power levels of interest in practice, and the power curve of the unadjusted analysis is obtained from f_{EFF}=1. FIG. 2 can provide support that a smaller f_{EFF} is capable of yielding higher power.

### Inferring Causal Estimands

As illustrated above, logistic regression models adjusted with prognostic scores can prospectively calculate sample size reductions and power gains in the planning stage of RCTs based on hypothesis tests for H₀: β₁=0 versus

$${H}_{0}^{*} : {B}_{1}^{*} = 0 .$$

However, it is also important to evaluate the frequentist properties of inferences for risk difference Δ_{RD}, relative risk Δ_{RR}, and odds ratio Δ_{OR} that are obtained via g-computation in adjusted logistic regression models. The efficiency factor can be utilized in these evaluations based on two connections between the logistic regression coefficients and these estimands. First, the null hypothesis H₀: β₁=0 may imply specific values for the estimands. Specifically, the null hypothesis H₀: β₁=0 generally leads to Δ_{RD}=0, Δ_{RR}=1, and Δ_{OR}=1. Second, hypothesis tests for the causal estimands in a logistic regression model adjusted by prognostic scores can be performed by utilizing g-computation with the coefficients in the model described in equation (1). In many embodiments, the efficiency factor is central to sample size calculations and power evaluations for the test of H₀: β₁=0 versus

$${H}_{0}^{*} : {β}_{1}^{*} = 0 ,$$

and consequently, it is an important consideration of tests for H₀: Δ_{RD}=0, H₀: Δ_{RR}=1, and H₀: Δ_{OR}=1 when model (1) is the true data generating mechanism.

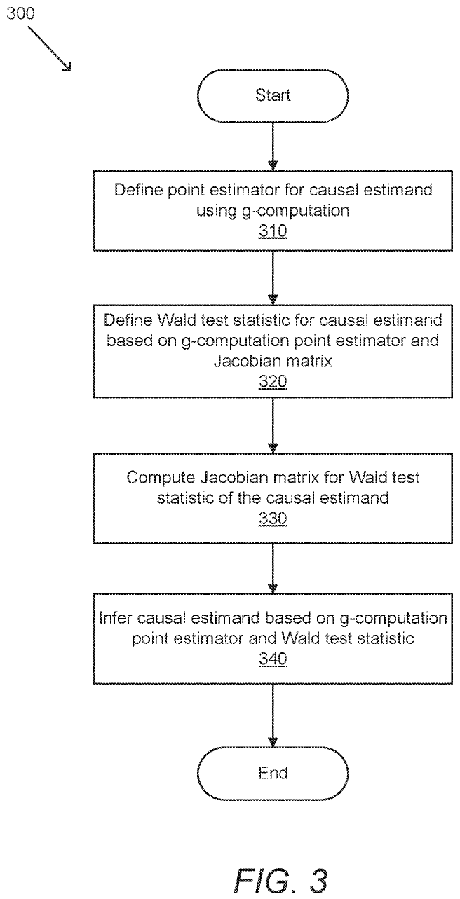

The Wald test can be an effective tool to test the significance of the treatment effect. The Wald test statistic enables one to estimate the efficiency factor based on the observed data in an RCT. FIG. 3 illustrates a process for inferring causal estimands in accordance with an embodiment of the invention. Process 300 defines (310) a point estimator for a causal estimand using G-computation. In many embodiments, Wald tests for Δ_{RD}, Δ_{RR}, and Δ_{OR} are directly derived based on the combination of the Delta method with g-computation for the fitted adjusted logistic regression model. The g-computation point estimator of each causal estimand can be obtained via a transformation G: [symbol] ³→ [symbol] of the estimators \hat{θ}ₙ=(\hat{β}₀, \hat{β}₁, \hat{β}₂)^{T} from the fitted model (1).

Process 300 defines (320) the Wald test statistic of the causal estimand based on the g-computation point estimator and a Jacobian matrix. The Wald test statistic may be defined using the combination of the Delta Method with the transformation according to

$$W = {n}^{1 / 2} G ( {\hat{θ}}_{n} ) {\{ {J}^{⊤} {\hat{V}}_{n} J \}}^{- 1 / 2} .$$

Process 300 computes (330) the Jacobian matrix for the Wald test statistic of the causal estimand. In several embodiments, the Jacobian matrices are a 3×1 Jacobian associated with the transformation G.

Process 300 infers (340) the causal estimand based on the g-computation point estimator and the Wald test statistic. By considering the risk difference and the natural logarithm of the relative risk, the transformations for Δ_{RD} and log (Δ_{RR}) are

$${G}_{RD} ( {\hat{β}}_{0} , {\hat{β}}_{1} , {\hat{β}}_{2} ) = \frac{1}{n} {\sum }_{i = 1}^{n} \{ \frac{exp ( {\hat{β}}_{0} + {\hat{β}}_{1} + {\hat{β}}_{2} {m}_{i} )}{1 + exp ( {\hat{β}}_{0} + {\hat{β}}_{1} + {\hat{β}}_{2} {m}_{i} )} \} - \frac{1}{n} {\sum }_{i = 1}^{n} \{ \frac{exp ( {\hat{β}}_{0} + {\hat{β}}_{2} {m}_{i} )}{1 + exp ( {\hat{β}}_{0} + {\hat{β}}_{2} {m}_{i} )} \} ,$$

$${G}_{log ( RR )} ( {\hat{β}}_{0} , {\hat{β}}_{1} , {\hat{β}}_{2} ) = log [ \frac{1}{n} {\sum }_{i = 1}^{n} \{ \frac{exp ( {\hat{β}}_{0} + {\hat{β}}_{1} + {\hat{β}}_{2} {m}_{i} )}{1 + exp ( {\hat{β}}_{0} + {\hat{β}}_{1} + {\hat{β}}_{2} {m}_{i} )} \} ] - log [ \frac{1}{n} {\sum }_{i = 1}^{n} \{ \frac{exp ( {\hat{β}}_{0} + {\hat{β}}_{2} {m}_{i} )}{1 + exp ( {\hat{β}}_{0} + {\hat{β}}_{2} {m}_{\dot{t}} )} \} ] .$$

The corresponding Jacobian for the risk difference transformation can be expressed as:

$${J}_{RD} = ( \begin{matrix}\frac{\partial {G}_{RD}}{\partial {\hat{β}}_{0}} \\ \frac{\partial {G}_{RD}}{\partial {\hat{β}}_{1}} \\ \frac{\partial {G}_{RD}}{\partial {\hat{β}}_{2}}\end{matrix} ) = ( \begin{matrix}\frac{1}{n} {\sum }_{i = 1}^{n} [ \frac{exp ( {\hat{β}}_{0} + {\hat{β}}_{1} + {\hat{β}}_{2} {m}_{i} )}{{\{ 1 + exp ( {\hat{β}}_{0} + {\hat{β}}_{1} + {\hat{β}}_{2} {m}_{i} ) \}}^{2}} ] - \frac{1}{n} {\sum }_{i = 1}^{n} [ \frac{exp ( {\hat{β}}_{0} + {\hat{β}}_{2} {m}_{i} )}{{\{ 1 + exp ( {\hat{β}}_{0} + {\hat{β}}_{2} {m}_{i} ) \}}^{2}} ] \\ \frac{1}{n} {\sum }_{i = 1}^{n} [ \frac{exp ( {\hat{β}}_{0} + {\hat{β}}_{1} + {\hat{β}}_{2} {m}_{i} )}{{\{ 1 + exp ( {\hat{β}}_{0} + {\hat{β}}_{1} + {\hat{β}}_{2} {m}_{i} ) \}}^{2}} ] \\ \frac{1}{n} {\sum }_{i = 1}^{n} [ \frac{{m}_{i} exp ( {\hat{β}}_{0} + {\hat{β}}_{1} + {\hat{β}}_{2} {m}_{t} )}{{\{ 1 + exp ( {\hat{β}}_{0} + {\hat{β}}_{1} + {\hat{β}}_{2} {m}_{i} ) \}}^{2}} ] - \frac{1}{n} {\sum }_{i = 1}^{n} [ \frac{{m}_{i} exp ( {\hat{β}}_{0} + {\hat{β}}_{2} {m}_{i} )}{{\{ 1 + exp ( {\hat{β}}_{0} + {\hat{β}}_{2} {m}_{i} ) \}}^{2}} ]\end{matrix} ) .$$

To simplify the notation, the Jacobian may be defined as:

$${J}_{01} = \frac{1}{n} {\sum }_{i = 1}^{n} [ \frac{exp ( {\hat{β}}_{0} + {\hat{β}}_{1} + {\hat{β}}_{2} {m}_{i} )}{{\{ 1 + exp ( {\hat{β}}_{0} + {\hat{β}}_{1} + {\hat{β}}_{2} {m}_{i} ) \}}^{2}} ] , {J}_{02} = \frac{1}{n} {\sum }_{\dot{t} = 1}^{n} [ \frac{exp ( {\hat{β}}_{0} + {\hat{β}}_{2} {m}_{i} )}{{\{ 1 + exp ( {\hat{β}}_{0} + {\hat{β}}_{2} {m}_{i} ) \}}^{2}} ] ,$$

$${J}_{21} = \frac{1}{n} {\sum }_{i = 1}^{n} [ \frac{{m}_{i} exp ( {\hat{β}}_{0} + {\hat{β}}_{1} + {\hat{β}}_{2} {m}_{i} )}{{\{ 1 + exp ( {\hat{β}}_{0} + {\hat{β}}_{1} + {\hat{β}}_{2} {m}_{i} ) \}}^{2}} ] , {J}_{22} = \frac{1}{n} {\sum }_{\dot{t} = 1}^{n} [ \frac{{m}_{i} exp ( {\hat{β}}_{0} + {\hat{β}}_{2} {m}_{i} )}{{\{ 1 + exp ( {\hat{β}}_{0} + {\hat{β}}_{2} {m}_{i} ) \}}^{2}} ] ,$$

$$such that$$

$${J}_{RD} = ( \begin{matrix}\frac{\partial {G}_{RD}}{\partial {\hat{β}}_{0}} \\ \frac{\partial {G}_{RD}}{\partial {\hat{β}}_{1}} \\ \frac{\partial {G}_{RD}}{\partial {\hat{β}}_{2}}\end{matrix} ) = ( \begin{matrix}{J}_{01} - {J}_{02} \\ {J}_{01} \\ {J}_{21} - {J}_{22}\end{matrix} ) .$$

Similarly, the Jacobian for the natural logarithm of relative risk can be defined as:

$${J}_{log ( RR )} = ( \begin{matrix}\frac{\partial {G}_{log ( RR )}}{\partial {\hat{β}}_{0}} \\ \frac{\partial {G}_{log ( RR )}}{\partial {\hat{β}}_{1}} \\ \frac{\partial {G}_{log ( RR )}}{\partial {\hat{β}}_{2}}\end{matrix} ) = ( \begin{matrix}{[ \frac{1}{n} {\sum }_{i = 1}^{n} \{ \frac{exp ( {\hat{β}}_{0} + {\hat{β}}_{1} + {\hat{β}}_{2} {m}_{i} )}{1 + exp ( {\hat{β}}_{0} + {\hat{β}}_{1} + {\hat{β}}_{2} {m}_{i} )} \} ]}^{- 1} {J}_{01} {- [ \frac{1}{n} {\sum }_{i = 1}^{n} \{ \frac{exp ( {\hat{β}}_{0} + {\hat{β}}_{2} {m}_{i} )}{1 + exp ( {\hat{β}}_{0} + {\hat{β}}_{2} {m}_{i} )} \} ]}^{- 1} {J}_{02} \\ {[ \frac{1}{n} {\sum }_{i = 1}^{n} \{ \frac{exp ( {\hat{β}}_{0} + {\hat{β}}_{1} + {\hat{β}}_{2} {m}_{i} )}{{\{ 1 + exp ( {\hat{β}}_{0} + {\hat{β}}_{1} + {\hat{β}}_{2} {m}_{i} ) \}}^{2}} \} ]}^{- 1} {J}_{01} \\ {[ \frac{1}{n} {\sum }_{i = 1}^{n} \{ \frac{exp ( {\hat{β}}_{0} + {\hat{β}}_{1} + {\hat{β}}_{2} {m}_{t} )}{1 + exp ( {\hat{β}}_{0} + {\hat{β}}_{1} + {\hat{β}}_{2} {m}_{i} )} \} ]}^{- 1} {J}_{21} {- [ \frac{1}{n} {\sum }_{i = 1}^{n} \{ \frac{exp ( {\hat{β}}_{0} + {\hat{β}}_{2} {m}_{i} )}{1 + exp ( {\hat{β}}_{0} + {\hat{β}}_{2} {m}_{i} )} \} ]}^{- 1} {J}_{22}\end{matrix} ) .$$

The transformations and Jacobians under the unadjusted model can be calculated in a similar manner as for the adjusted model. Calculations for the natural logarithm of the odds ratio for the prognostic score can also be performed in a similar manner.

In addition to tests, confidence intervals of the causal estimands can be calculated using either the variance estimators from the Delta method or the nonparametric bootstrap. Although computationally more intensive, the nonparametric bootstrap is agnostic to whether the analysis model underlies the true data-generating mechanism. In several embodiments, G-computation can be combined with the nonparametric bootstrap to construct confidence intervals for the estimands. This combination corresponds with regulatory guidance on the use of the bootstrap for analyses that involve covariate adjustment.

### Correcting for Model Misspecification

The validity of the Wald test for H₀: β₁=0 in a model adjusted with prognostic scores, as well as of the efficiency factor based on equation (9), may be affected as a result of model misspecification. Three types of model misspecifications are common in practice: the omission of an important covariate, a shift in the prognostic scores, and random errors in the prognostic scores. In many embodiments, the efficiency factor remains valid when there is an omission of an important covariate or a shift in the prognostic scores. For random errors in prognostic scores, the efficiency factor may be adjusted to yield more accurate prospective predictions of gains that can result from adjusting logistic regression models based on prognostic scores.

When there is an omission of an important covariate from both the procedure for constructing the prognostic scores and from direct adjustments in the logistic regression model, prognostic covariate adjustment may still provide a partial adjustment for the covariates, as the omitted covariate is neither contained in the prognostic score nor as a predictor variable in the logistic regression model. In several embodiments, logistic regression models that utilize only a partial adjustment can produce valid tests for the null hypothesis of the treatment effect. In addition, the efficiency factor based on equation (9) can remain valid because robust estimates of variance were used in the derivation.

There are cases where the prognostic score mᵢ that is utilized in the prognostic covariate adjustment may not be the true predictor underlying the data generation mechanism, but instead, a shifted version of the prognostic score \tilde{m}ᵢ=mᵢ+b is the true predictor for data generation. The shift can be defined according to the bias term b. In several embodiments, as the adjusted model has an intercept term, the parameter β₀ absorbs the bias b. Hence, the Wald test for H₀: β₁=0 in the adjusted model analysis can remain valid. In some embodiments, the calculation of the efficiency factor based on the mᵢ can consequently be similar to the true efficiency factor that would have been calculated if the \tilde{m}ᵢ were observable. This is because, although the mᵢ and \tilde{m}ᵢ differ, the corresponding values for the participants' probabilities of an event under control, i.e., the μ_{0,i}, may not differ as much after the logistic transformation. Hence, the E(μ_{0,i}) can be fairly similar when calculated using either mᵢ or \tilde{m}ᵢ. In addition, as the bias term is additive, the variances of the mᵢ and \tilde{m}ᵢ may be the same, and so the corresponding values of Var(μ_{0,i}) may also be similar.

The third type of model misspecification corresponds to the case in which the observed prognostic scores mᵢ differ from the true prognostic scores \tilde{m}ᵢ underlying the data generation mechanism by random error terms, i.e., \tilde{m}ᵢ=mᵢ+δᵢ for random variables δᵢ. This case corresponds to logistic regression with errors in variables. Previous investigations in this domain have not considered the validity of statistical tests for the coefficients but instead primarily focused on adjusting the maximum likelihood estimators of the logistic regression coefficients so that they are asymptotically unbiased. The efficiency factor based on equation (9) may not be directly applicable in the case of random errors in the prognostic scores. This is because the efficiency factor involves the variance of the μ_{0,i}, and the observed prognostic scores mᵢ that are used in the adjusted analysis to estimate this variance can contain spurious variability. Hence, Var(μ_{0,i}) will be overestimated, and the efficiency factor will overestimate the benefit of adjustment by the prognostic score. In several embodiments, the Wald test maintains its nominal significance level in this case. To address this overestimation and more accurately estimate the gains of adjusted models in this case, in many embodiments, equation (9) can be adjusted by using the fact that the squared correlation between the μ_{0,i} that are calculated based on mᵢ, and the \tilde{μ}_{0,i} that are calculated based on the \tilde{m}ᵢ, correspond to the percentage of variance in \tilde{μ}_{0,i} that can be explained by μ_{0,i}. Hence, the efficiency factor from equation (9) can also be adjusted by this correlation according to

$${\tilde{f}}_{EFF} = \sqrt{1 - \frac{Var ( {μ}_{0 , i} ) Corr {( {\tilde{μ}}_{0 , i} , {μ}_{0 , i} )}^{2}}{E ( {μ}_{0 , i} ) \{ 1 - E ( {μ}_{0 , i} ) \}}} . \qquad ( 14 )$$

to remedy the risk of overconfidence in adjusted models in the case of random errors in the prognostic scores. The correlation between μ_{0,i} and \tilde{μ}_{0,i} may be unknown in practice, and one straightforward approach to estimate this correlation can be by using the concordance index between the observed and predicted binary outcomes.

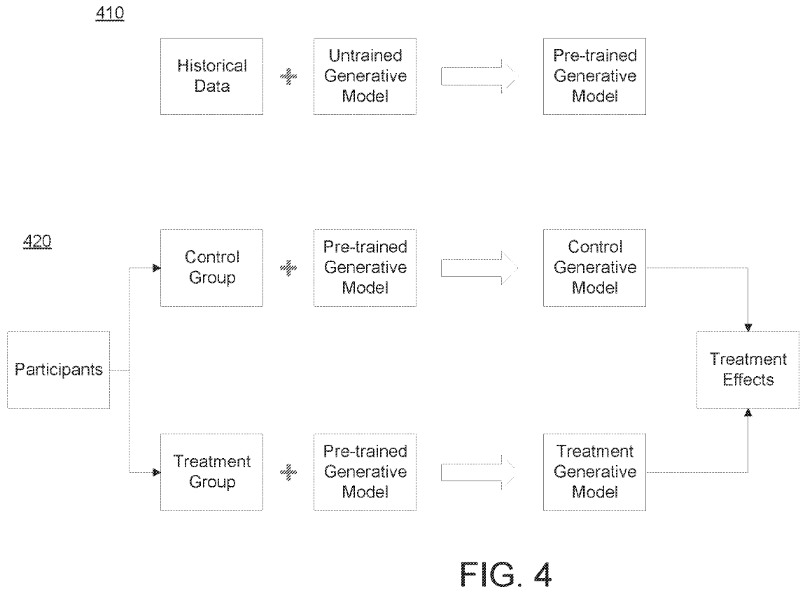

An example of using generative models to estimate treatment effects in accordance with an embodiment of the invention is illustrated in FIG. 4. In the first stage 410, an untrained generative model of the control condition is trained using historical data to become a pre-trained generative model. Historical data may include, but are not limited to, data from previously completed clinical trials, electronic health records, and/or other studies. In several embodiments, the untrained generative model may be used to generate digital twins to represent potential participants. In the second stage 420, a participant population is randomly divided into a control group and a treatment group as part of a randomized controlled trial. Participants from the population can be randomized into the control and treatment groups with unequal randomization in accordance with a variety of embodiments of the invention. In stage 420, the pre-trained generative model can take as input the baseline covariates of the participants in the treatment and control groups to generate the digital twin distributions for the participants in the RCT. In several embodiments, pre-trained generative models that take the baseline covariates of the participants in the control and treatment groups as inputs may become the control generative models and treatment generative models, respectively. In certain embodiments, control and treatment generative models can be based on a pre-trained generative model but can be additionally trained to reflect new information from the RCT. Outputs from the control generative models can then be utilized to estimate the treatment effects. In several embodiments, Bayesian methods and/or the bootstrap may be used to estimate uncertainties in the treatment effect estimators and decision rules based on p-values and/or posterior probabilities may be applied. Trial design may be optimized based on the generative models and associated performances of the estimations.

### Hardware Implementation

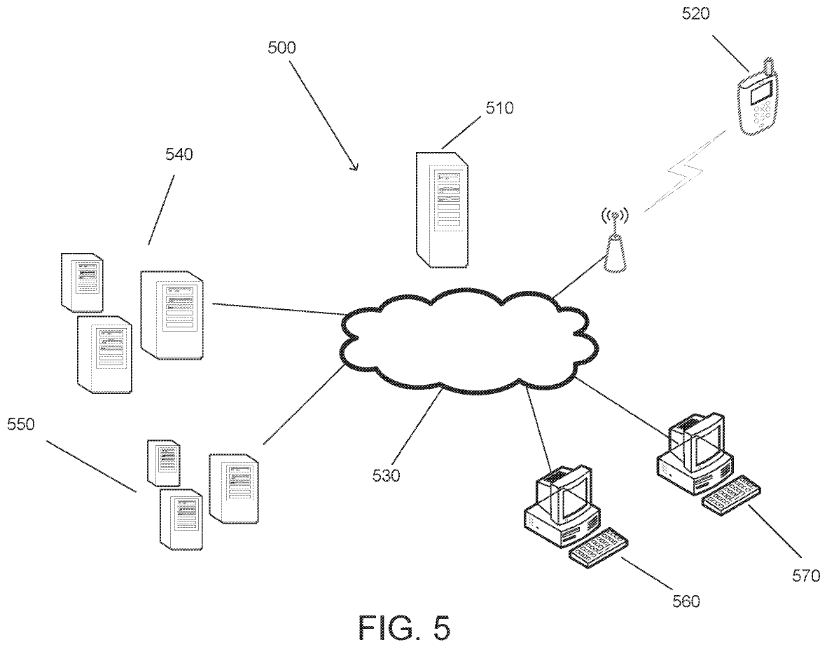

An example of a network in which the processes described above can be implemented in accordance with an embodiment of the invention is illustrated in FIG. 5. In many embodiments, network 500 includes a communication network 530. Communication network 530 may be a network such as the Internet that allows devices connected to the network 530 to communicate with other connected devices. In a number of embodiments, server systems 540 and 550 can be connected to the network 530. According to various embodiments of the invention, each of the server systems 540 and 550 may be a group of one or more servers communicatively connected to one another via internal networks that execute processes that provide cloud services to users over the network 530. For purposes of this discussion, cloud services are one or more applications that are executed by one or more server systems to provide data and/or executable applications to devices over a network.

The server systems 540 and 550 are shown to each have three servers in the internal network. However, the server systems 540 and 550 may include any number of servers, and any additional number of server systems may be connected to the network 530 to provide cloud services. In some embodiments, there may only be a single server 510 that is connected to network 530 to provide services to users. In accordance with various embodiments of this invention, a computing system that uses systems and methods that design RCTs using prognostic covariate adjustment in logistic regression models in accordance with an embodiment of the invention may be provided by a process being executed on a single server system and/or a group of server systems communicating over network 530.

Users may use personal devices 560 and 570 that connect to the network 530 to perform processes that design RCTs using prognostic covariate adjustment in logistic regression models in accordance with various embodiments of the invention. In the shown embodiment, the personal devices 560 and 570 are shown as desktop computers that are connected via a conventional “wired” connection to the network 530. However, personal devices 560 and 570 may be a desktop computer, a laptop computer, a smart television, an entertainment gaming console, or any other device that connects to the network 530 via a “wired” connection. Mobile device 520 can connect to network 530 using a wireless connection. A wireless connection may be a connection that uses Radio Frequency (RF) signals, Infrared signals, or any other form of wireless signaling to connect to the network 530. In the example of this figure, the mobile device 520 is a mobile telephone. However, mobile device 520 may be a mobile phone, Personal Digital Assistant (PDA), a tablet, a smartphone, or any other type of device that connects to network 530 via wireless connection without departing from this invention.

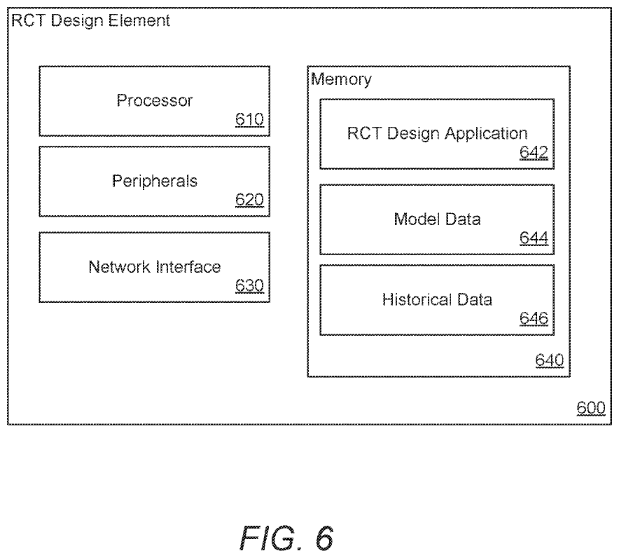

An example of an RCT design element that processes described above can be implemented on in accordance with an embodiment of the invention is illustrated in FIG. 6. RCT design element 600 includes a network interface 630 that can receive external data, and a memory 640 to store the various types of data including model data 644 and historical data 646. Processor 610 may execute RCT design application 642 to design RCTs using prognostic covariate adjustment in logistic regression models in accordance with several embodiments of the invention. One skilled in the art will recognize that the computing system may exclude certain components and/or include other components that are omitted for brevity without departing from this invention.

In many embodiments, processor 610 can include a processor, a microprocessor, a controller, or a combination of processors, microprocessors, and/or controllers that perform instructions stored in memory 640 to manipulate historical data stored in the memory. Processor instructions can configure the processor 610 to perform processes in accordance with certain embodiments of the invention. In various embodiments, processor instructions can be stored on a non-transitory machine readable medium.

Although a specific example of an RCT design element is illustrated in this figure, any of a variety of treatment effects estimation elements can be utilized to perform processes for designing RCTs using prognostic covariate adjustment in logistic regression models similar to those described herein as appropriate to the requirements of specific applications in accordance with embodiments of the invention.

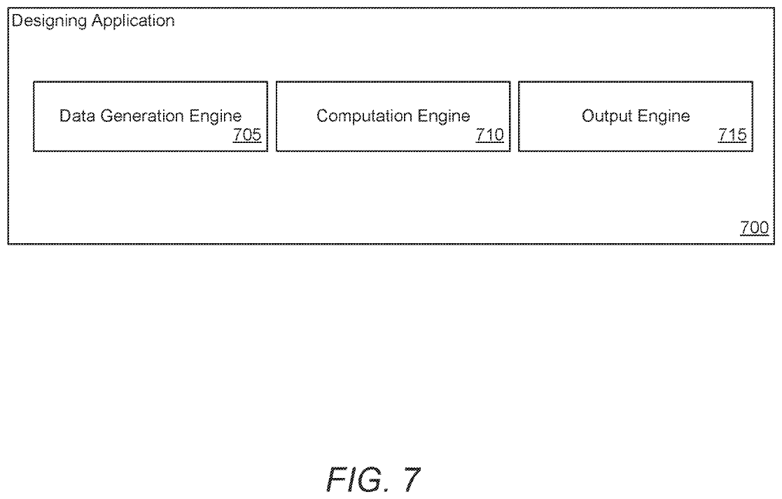

An example of a design application that executes instructions to design RCTs using prognostic covariate adjustment in logistic regression models in accordance with an embodiment of the invention is illustrated in FIG. 7. In several embodiments, RCT design application may include a data generation engine 705, a computation engine 710, and an output engine 715. Data generation engine 705 in accordance with various embodiments of the invention can be used to generate digital twins for use as trial participants in the designing stage of an RCT. In several embodiments, computation engine 710 can be used to perform the various computations necessary for RCT design using prognostic covariate adjustment as described above. In some embodiments, output engine 715 can be used to output the results of RCT design.

Although a specific example of RCT design application is illustrated in this figure, any of a variety of RCT design applications can be utilized to perform processes for designing RCTs using prognostic covariate adjustment in logistic regression models similar to those described herein as appropriate to the requirements of specific applications in accordance with embodiments of the invention.

Although specific methods of designing RCTs using prognostic covariate adjustment in logistic regression models are discussed above, many different design methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for randomized controlled trial (RCT) design using prognostic covariate adjustment in logistic regression models, the method comprising:
generating a plurality of digital twin distributions for prospective trial participants;
generating a plurality of digital twins based on the generated digital twin distributions;
calculating prognostic scores for each participant based on their generated digital twins;
optimizing trial design based on the prognostic scores;
fitting a logistic regression model to observed data comprising the digital twins of the participants, actual outcomes of each participant, and the prognostic scores; and
estimating treatment effects based on the fitted model.

2. The method of claim 1, wherein the plurality of digital twin distributions are generated using models trained on historical patient data, wherein the plurality of digital twin distributions generates forecasts for prospective trial participants.

3. The method of claim 1, wherein the digital twin distributions are Bernoulli distributions.

4. The method of claim 1, wherein the prognostic score is an expectation of the digital twin distribution for a prospective trial participant.

5. The method of claim 1, further comprising computing a set of regression coefficient estimates of the logistic regression model based on the prognostic scores.

6. The method of claim 5, wherein the set of regression coefficients comprises a treatment indicator coefficient, and a prognostic score coefficient.

7. The method of claim 1, wherein the plurality of treatment effects comprises risk difference, risk ratio, and odds ratio.

8. The method of claim 1, wherein optimizing trial design further comprises reducing the sample size of the plurality of prospective trial participants and increasing the power of the logistic regression model.

9. The method of claim 1, wherein the prognostic score is calculated based on a set of baseline covariates.

10. The method of claim 1, wherein the prognostic score associates a set of baseline covariates with a probability of an event under control for each prospective trial participant.

11. The method of claim 1, wherein the logistic regression model further comprises a treatment indicator wherein prospective trial participants are randomly assigned to either a treatment group or a control group.

12. The method of claim 8, wherein the sample size of the plurality of prospective trial participants comprises is reduced based on an efficiency factor.

13. The method of claim 12, wherein the efficiency factor is determined based on a bias factor and an asymptotic relative efficiency of the logistic regression model.

14. The method of claim 13, wherein the efficiency factor is estimated based on a ratio of Wald test statistics for an unadjusted logistic regression model and an adjusted logistic regression model.

15. The method of claim 14, wherein the ratio of Wald test statistics is predicted based on the variance and an expectation of the probability of an event for a prospective trial participant under control.

16. The method of claim 8, wherein the power of the logistic regression model is determined based on an unadjusted Wald test statistic for null hypothesis on a treatment assignment coefficient and an efficiency factor.

17. The method of claim 7, wherein the plurality of treatment effects is determined based on a combination of Delta method and G-computation.

18. The method of claim 7, wherein a point estimator for each of the plurality of treatment effects can be computed using G-computation.

19. The method of claim 1, wherein the prognostic scores are calculated based on an external and historical control dataset.

20. A non-transitory machine readable medium containing processor instructions for ROT design using prognostic covariate adjustment in logistic regression models, where execution of the instructions by a processor causes the processor to perform a process that comprises:
generating a plurality of digital twin distributions for prospective trial participants; generating a plurality of digital twins based on the generated digital twin distributions;
calculating prognostic scores for each participant based on their generated digital twins, wherein the prognostic score is an expectation of the digital twin distribution for a prospective trial participant;
optimizing trial design based on the prognostic scores;
fitting a logistic regression model to observed data comprising the digital twins of the participants, actual outcomes of each participant, and the prognostic scores;
computing a set of regression coefficient estimates of the logistic regression model based on the prognostic scores, wherein the set of regression coefficients comprises a treatment indicator coefficient, and a prognostic score coefficient; and
estimating treatment effects based on the fitted model.
